(19) United States
(12) Patent Application Publication
(10) Pub. No.: US 2007/0265565 A1
(43) Pub. Date: Nov. 15, 2007

(54) Mesh-Reinforced Catheter Balloons and Methods for Making the Same

(75) Inventor: David Johnson, Turloughmore (IE)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/383,240

(22) Filed: May 15, 2006

(51) Int. Cl. A61M 29/00; A61M 31/00; A61M 37/00

(52) U.S. Cl. 604/103.06; 604/96.01

(57) ABSTRACT

Disclosed herein are mesh-reinforced catheter balloons and methods for making the same. Specifically, the mesh-reinforced catheter balloons comprise a low compliance balloon material wherein the mesh is a highly oriented version of the material forming the balloon.

### FIELD OF THE INVENTION

The present invention relates to mesh-reinforced catheter balloons and methods for making the same. Specifically, the mesh-reinforced catheter balloons comprise a low compliance balloon material wherein the mesh is a highly oriented version of the material forming the balloon.

### BACKGROUND OF THE INVENTION

Balloon catheters are commonly used in surgical procedures to contribute to the treatment of vessel abnormalities. For example, balloon catheters can be used to dilate or remove constrictions in vessels or to deliver and deploy other devices, such as stents, within vessels. When used to treat a vessel constriction, for example, the balloon catheter with a stent on its distal end is inserted within the patient and navigated through the vessel to the site of the blockage. The balloon at the distal end of the catheter is then inflated, causing the balloon to increase in diameter. This increase in diameter serves both to open the blocked vessel and to deploy the stent. Once the blockage is opened and the stent is deployed, the balloon is deflated and removed from the patient.

Balloon catheters may employ various balloon materials depending on the application for which they are used. For example, embolectomy balloon catheters utilize elastomeric balloon materials such as latex or silicone because in such procedures there is no need for the use of high inflation pressures (embolectomy procedures remove abnormal particles, such as air bubbles or clot particles circulating in the blood stream). Angioplasty balloon catheters, on the other hand, utilize relatively inelastic materials such as polyester or nylon because in such procedures the application of high inflation pressure is often required.

Elastomeric and inelastic balloon materials each have advantages and drawbacks. While elastomeric materials are generally soft and conformable, they lack strength and exhibit continuous diameter growth with the application of increasing inflation pressure until rupture occurs. Elastomeric balloon materials are referred to as compliant. Inelastic balloon materials have very predictable diameter growth characteristics, and distend very little beyond their intended diameter with the application of increasing inflation pressure. Inelastic balloon materials are referred to as non-compliant or semi-compliant depending on their stiffness.

Weaknesses in the walls of both types of balloons can result in a risk that the balloon will burst during inflation, notably where high inflation pressures are used. The problems due to potential weaknesses in the balloon walls are accentuated when they are used to expand a stent because stents can often have relatively sharp edges that can snag portions of the balloon wall during deployment. In this situation, replacement balloons must be used, increasing the time of the procedure (during which time arterial blood flow is restricted), thus increasing patient risk and trauma, and incurring significant additional cost.

To address the problem of rupture, many balloons are reinforced with a mesh. However, balloons reinforced with mesh to date have all been semi-compliant in nature which is not desirable in high pressure inflation applications. Further, to date, mesh-reinforced semi-compliant balloons all include an elastomeric matrix phase material (such as polyurethane, latex or silicone). Inclusion of this elastomeric matrix phase material is not ideal as it is costly to manufacture, results in excessively thick/stiff necks that are difficult to bond to other catheter materials and undergoes breakage at critical strain points resulting in non-uniform expansion of the balloon (and as a result the stent) and the loss of the balloon's original dimension, form and shape during subsequent inflation/deflation cycles.

Further, in conventional thermoplastic balloon processing, the extent to which the balloon tubing is radially stretched determines the hoop strength of the balloon and therefore the burst pressure for any given thickness. A limitation of this method is that radial stretching also tends to thin the balloon thereby offsetting potential gains in burst pressure. Additionally, this type of stretching can introduce microscopic/pinhole defects, which can act as precursors to failure thus limiting the balloon wall strength.

Thus, none of these presently available catheter balloons offer a reinforced non-compliant/low compliant balloon material. Moreover, none of these presently available balloons selectively reinforce the balloon with a more highly orientated version of the same material. Reinforcing with a more highly orientated version of the same/similar material would significantly strengthen the wall of the balloon, thereby allowing a thinner more flexible continuous phase to be used. This approach recognizes and applies a feature of polymer behavior, whereby the theoretical strength of the material is only approached when the material is highly orientated as in the case of fibrous mesh. A major advantage of this approach is that higher radial strength is realized without the need for higher stretch/radial ratios, which can introduce microscopic or pinhole defects. In contrast, in an orientated mesh, not only is orientation optimized but also intrinsic voids/pinhole defects are greatly reduced or eliminated. The present invention takes advantage of these approaches and characteristics to provide an improved catheter balloon that does not suffer drawbacks inherent with those of the prior art.

### SUMMARY OF THE INVENTION

The present invention provides a balloon with high burst strength with reduced likelihood of fragmentation. The present invention increases burst strength and reduces the likelihood of fragmentation by incorporating a reinforcement mesh into the wall of a low compliance balloon with the reinforcement mesh comprising a more highly orientated version of the balloon material. Such reinforcement can increase the load carrying capacity of the balloon material and inhibit/terminate crack growth. Application of fibrous mesh can also ensure that the balloon supports applied stresses and resultant longitudinal and hoop stresses in the most efficient manner possible. Importantly, when these features are created through the use of mesh reinforcement with a highly oriented version of the balloon material, a higher radial strength can be realized without the need for higher stretch/radial ratios.

Specifically, one embodiment according to the present invention is a catheter balloon comprising a mesh-reinforced low compliant balloon material wherein the mesh is a highly oriented version of the balloon material.

In another embodiment of the catheter balloons according to the present invention, the mesh and the balloon material are from the same polymer family or are the same polymer. In specific embodiments appropriate mesh and the balloon materials are selected from the group consisting of polyamide, co-polyamide, aromatic polyamide, polyester, polyethylene and ultra-high molecular weight polyethylene (UHMWPE). In a particular embodiment of the catheter balloons, the mesh is a polyamide mesh.

In embodiments of the catheter balloons, the mesh can be added to the surface of the balloon, the interior of the balloon or both. In certain embodiments, the mesh is added to the surface, interior or surface and interior of a polyamide or co-polyamide balloon.

In additional embodiments of the catheter balloons, the catheter balloon comprises a high strength polyamide fiber matrix wherein the strength of the balloon is derived from the fibrous phase and the flexibility of the balloon is derived from the balloon's thin wall continuous matrix phase.

The present invention also includes methods. In one method according to the present invention the method comprises providing a catheter balloon comprising a mesh-reinforced low compliant balloon material wherein the mesh is a highly oriented version of the balloon material.

In another embodiment of the methods according to the present invention, the balloon material is selected from the group consisting of polyamide, co-polyamide, aromatic polyamide, polyester, polyethylene and ultra-high molecular weight polyethylene (UHMWPE).

In yet another embodiment, the method comprises placing a mesh pre-form over balloon tubing; blowing the balloon tubing inside a balloon mold to embed the mesh into the outside surface of the balloon; removing the mesh-embedded balloon from the mold; and adding an overcoat to the mesh attached to the surface of the balloon to affix the mesh in place; thereby providing a catheter balloon comprising a mesh-reinforced low compliant balloon material wherein the mesh is a highly oriented version of the balloon material.

In certain embodiments, the mesh pre-form and/or the balloon material comprises a polymer selected from the group consisting of polyamide, co-polyamide, aromatic polyamide, polyester, polyethylene and ultra-high molecular weight polyethylene (UHMWPE). In particular embodiments of the methods, the mesh pre-form and/or the balloon material comprise polyamide.

An additional embodiment of the methods comprises blowing balloon tubing against a mesh structure inside a mold to form a mesh footprint on the outside surface of the balloon; removing the imprinted balloon from the mold; attaching mesh to the surface of the imprinted balloon; and adding an overcoat to the mesh to affix the mesh to the surface of the imprinted balloon; thereby providing a catheter balloon comprising a mesh-reinforced low compliant balloon material wherein the mesh is a highly oriented version of the balloon material.

Another embodiment of the methods comprises placing an ultra-high molecular weight fibrous mesh pre-form over balloon tubing in a mold; blowing the inside of the balloon tubing to form a balloon shape and to fuse the mesh pre-form into the balloon material; and removing the formed balloon with the fused mesh pre-form from the mold thereby providing a catheter balloon comprising a mesh-reinforced low compliant balloon material wherein the mesh is a highly oriented version of the balloon material. In another embodiment of the methods the mesh pre-form and the balloon tubing comprise a material selected from the group consisting of polyamide and polyethylene.

The last presently described method comprises injection molding a balloon pre-form with a mesh insert; and blowing the balloon pre-form using a stretch blow molding process thereby providing a catheter balloon comprising a mesh-reinforced low compliant balloon material wherein the mesh is a highly oriented version of the balloon material.

### Definition of Terms

Prior to setting forth embodiments according to the present invention, it may be helpful to an understanding thereof to set forth definitions of certain terms that will be used hereinafter.

As used herein, “highly oriented” includes polymer chains having a high degree of orientation, whereby the polymer molecules are preferentially orientated in the direction of stretching or in the extrusion machine direction. A material that is highly oriented according to the present invention will have a tensile modulus that is at least about 15 times higher than a non-highly oriented version of the same material or a tensile strength that is at least about 35 times higher than a non-highly oriented version of the same material. For further discussion of these concepts, see Krom et al., 22 Polymer Testing 463-470 (2003) which is incorporated by reference herein for its teachings regarding orientation and tensile measurements.

As used herein, “reinforcing phase” includes any suitable configuration of reinforcement that strengthens or supports the continuous matrix phase.

As used herein, “highly orientated reinforcing phase” includes any suitable configuration of reinforcement with a high degree of polymer molecular orientation as described above.

As used herein, “continuous matrix phase” includes the principal balloon material to which the reinforcing phase is affixed.

As used herein, “mesh pre-form” includes a tubular pre-form of mesh reinforcement, or any other suitable configuration, that is fitted over balloon tubing prior to the balloon forming process.

As used herein, “ultra-high molecular weight fibrous pre-form” include those having a molecular weight of greater than about 1,000,000.

### DETAILED DESCRIPTION

Balloons according to the present invention offer a reinforced non-compliant/low compliant balloon material. These balloons have high burst strength, a predetermined maximum diameter and the ability to recover to substantially pre-inflation size following deflation. Should rupture of these reinforced balloons nonetheless occur, the presence of the balloon reinforcing mesh can serve to contain fragments of the ruptured balloon. Finally, balloons according to the present invention can also incorporate other optional features.

Balloons in accordance with the present invention reinforce the balloon with a mesh that is a more highly orientated version of the balloon material. Reinforcing with a more highly orientated version of the same/similar material significantly strengthens the wall of the balloon, thereby allowing a thinner more flexible continuous phase to be used. This approach recognizes and applies a feature of polymer behavior, whereby the theoretical strength of the material is only approached when the material is highly orientated as in the case of fibrous mesh. The present invention takes advantage of these approaches and characteristics to provide an improved catheter balloon that does not suffer drawbacks inherent with those of the prior art.

Balloons made in accordance with the present invention can be of any suitable size and shape having regard to the purpose and intended use of the balloon. In the majority of embodiments, the balloons will adopt a cylindrical configuration when deployed. In certain embodiments, the tubular portion of the balloon catheter will have an external diameter of from about 0.5 to about 1.5 millimeters. This diameter can also be larger. In general, and in certain embodiments, the external diameter of the deployed but unstretched balloon will be at least about 1.5 times that of the tubular portion of the catheter, for example from about 1 to about 10 millimeters. However, because balloons according to the present invention do not undergo significant wall thinning or axial movement during expansion, it is possible to form balloons with an external deployed diameter of about 20 to about 25 mm or more. Similarly, balloons according to the present invention can be of any suitable axial length including, up to about 300 mm.

In a first example of how balloons in accordance with the present invention can be manufactured, a polyamide mesh pre-form (without limitation, polyamide, co-polyamide or aromatic polyamide) is placed over polyamide balloon tubing or any other suitable/biocompatible balloon material. Next, the mesh is embedded into the outside surface of the balloon by blowing the tubing and mesh against the inside of the balloon mold. Subsequently, the mesh-embedded balloon is removed from the mold. Finally, an overcoat (without limitation, spray or dip coated) is placed onto the balloon to affix the mesh in place. In accordance with this and other presently described examples, other fibers, including without limitation, aromatic polyamide/polyester/ultra-high molecular weight polyethylene (UHMWPE) fibers could also be used. The end product of this method is a strong flexible balloon. In one embodiment the balloon can comprise a lubricious outer surface.

In a second example of how balloons in accordance with the present invention can be manufactured, polyamide balloon tubing is blown against a mesh structure inside a mold to form a mesh footprint on the outside surface of the balloon. Then the imprinted balloon is removed from the mold. Next, mesh is attached to the balloon surface and an overcoat is, without limitation, spray or dip coated onto the balloon to affix the mesh in place.

In a third example of how balloons in accordance with the present invention can be manufactured, a polyamide/ultra high molecular weight polyethylene fibrous mesh pre-form is placed over polyamide balloon tubing, or any other suitable balloon material. Next, tubing inside the balloon is blown to form a balloon shape and to fuse the mesh into the balloon material. Finally, the balloon with an integral mesh is removed from the mold.

In a fourth example of how balloons in accordance with the present invention can be manufactured, a balloon pre-form is injection molded with a mesh insert. Next, the balloon is blown using standard balloon stretch blow molding processes that are known to those of ordinary skill in the art.

Balloons made in accordance with the present invention comprise a mesh-reinforced low compliant balloon material wherein the mesh is a highly oriented version of the balloon material. The mesh material and the balloon material can be from the same polymer family or can be the same polymer. Non-limiting examples of polymers that can be used in accordance with the present invention include polyamide, co-polyamide, aromatic polyamide, polyester, polyethylene and ultra-high molecular weight polyethylene (UHMWPE).

Meshes of the currently described and claimed balloons can be added to the surface of a balloon, can be added to the interior of a balloon or can be added to the surface and interior of a balloon. In certain embodiments, the catheter balloons comprise a high strength polyamide fiber matrix wherein the strength of the balloon is derived from the fibrous phase and the flexibility of the balloon is derived from the balloon's thin wall continuous matrix phase.

Embodiments according to the present invention, especially those including mesh embedding, footprinting or fusing allow for the strengthening of balloon walls and therefore for an increase in burst pressure without a corresponding increase in wall thickness. Indeed, certain embodiments according to the present invention increase burst pressure with no corresponding increase in wall thickness.

Balloons made in accordance with the present invention can embody additional features to enhance their efficacy or ease of use. One non-limiting example of such a feature could include a lubricating adjuvant incorporated on the external surface of the balloon so that it is more readily fed through the insertion tube and blood vessel to the desired location in the body and can then be readily deployed without sticking in a furled configuration. Balloons made in accordance with the present invention can also be produced to be liquid tight or can be produced with one or more regions of porosity through which various therapeutic agents can be delivered. Balloons made in accordance with the present invention can also comprise any suitable number of layers.

In some embodiments, it may be desirable for balloons made in accordance with the present invention to shorten or lengthen as they are inflated. For example, if a balloon catheter is used to deploy a stent that shortens as it grows in diameter, it could be desirable for the balloon to shorten in unison with the stent during deployment. Conversely, in such an application of a balloon catheter it could also be desirable for the balloon to slightly lengthen during inflation to counteract the shortening of the stent being deployed. Both embodiments can be employed in accordance with the present invention.

The present invention is an improved balloon and balloon catheter device for use in a variety of surgical procedures. The balloon catheter device of the present invention comprises a catheter tube having a continuous lumen connected to an inflatable and deflatable balloon at one end of the catheter tube. The catheter tube may have additional lumens provided for other purposes. Balloons according to the present invention have a burst strength that is greater than that of conventional angioplasty catheter balloons.

The design and manufacture of catheter components and assemblies thereof is well known. Catheter members can be of any suitable material or combination of materials such as, but not limited to, silicone, polyurethane, nylon, polyethylene, various co-polymers such as PolyEther Block Amide (PEBA), or polytetrafluoroethylene (PTFE). In some embodiments catheter members can contain metallic elements such as, but not limited to, braids, hypodermic tubing and/or wires. Any suitable method can be employed to create the attachments between the various elements of the balloon catheter. Such methods can include, but are not limited to, the use of various adhesives or thermal bonding techniques.

Various techniques can also be employed to enhance the connection between balloon and the catheter member(s). For example, reinforcing bands made in any suitable configuration of any suitable material may be placed around the balloon at points where the balloon is attached to the catheter member(s). Alternatively, the regions of attachment may be wrapped by reinforcing filaments of any suitable material. Usage of thin films may also yield advantageous embodiments.

Unless otherwise indicated, all numbers expressing quantities of ingredients, properties such as molecular weight, reaction conditions, and so forth are to be understood as being modified in all instances by the term “about.” Accordingly, unless indicated to the contrary, the numerical parameters set forth are approximations that may vary depending upon the desired properties sought to be obtained. At the very least, each numerical parameter should at least be construed in light of the number of reported significant digits and by applying ordinary rounding techniques. Notwithstanding that the numerical ranges and parameters are approximations, the numerical values set forth in the specific examples are reported as precisely as possible. Any numerical value, however, inherently contains certain errors necessarily resulting from the standard deviation found in their respective testing measurements.

The terms “a” and “an” and “the” and similar referents used are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Embodiments according to this invention are described herein, variations of those embodiments will become apparent to those of ordinary skill in the art upon reading the foregoing description.

Furthermore, numerous references have been made to patents and printed publications throughout this specification. Each of the above cited references and printed publications are herein individually incorporated by reference in their entirety.

What is claimed is:

1. A catheter balloon comprising a mesh-reinforced low compliant balloon material wherein said mesh is a highly oriented version of said balloon material.

2. A catheter balloon according to claim 1 wherein said mesh and said balloon material are from the same polymer family.

3. A catheter balloon according to claim 1 wherein said mesh and said balloon material are the same polymer.

4. A catheter balloon according to claim 1 wherein said mesh and said balloon material are selected from one or more of the group consisting of polyamide, co-polyamide, aromatic polyamide, polyester, polyethylene and ultra-high molecular weight polyethylene (UHMWPE).

5. A catheter balloon according to claim 1 wherein said mesh is a polyamide mesh.

6. A catheter balloon according to claim 1 wherein said mesh is added to the surface of said balloon.

7. A catheter balloon according to claim 6 wherein said balloon is a polyamide or co-polyamide balloon.

8. A catheter balloon according to claim 1 wherein said mesh is added to the interior of said balloon.

9. A catheter balloon according to claim 8 wherein said balloon is a polyamide or co-polyamide balloon.

10. A catheter balloon comprising a high strength polyamide fiber matrix wherein said strength of said balloon is derived from the fibrous phase and the flexibility of said balloon is derived from said balloon's thin wall continuous matrix phase.

11. A method comprising providing a catheter balloon comprising a mesh-reinforced low compliant balloon material wherein said mesh is a highly oriented version of said balloon material.

12. A method according to claim 11 wherein said balloon material is selected from the group consisting of polyamide, co-polyamide, aromatic polyamide, polyester, polyethylene and ultra-high molecular weight polyethylene (UHMWPE).

13. A method according to claim 11 wherein said method comprises:
placing a mesh pre-form over balloon tubing;
blowing said balloon tubing inside a balloon mold to embed said mesh pre-form into the surface of the balloon;
removing the mesh-embedded balloon from said mold; and
adding an overcoat to said mesh embedded in said surface of said balloon to affix said mesh in place; thereby
providing a catheter balloon comprising a mesh-reinforced low compliant balloon material wherein said mesh is a highly oriented version of said balloon material.

14. A method according to claim 13 wherein said mesh pre-form comprises a polymer selected from the group consisting of polyamide, co-polyamide, aromatic polyamide, polyester, polyethylene and ultra-high molecular weight polyethylene (UHMWPE).

15. A method according to claim 14 wherein said balloon material comprises polyamide.

16. A method according to claim 11 wherein said method comprises:
blowing balloon tubing against a mesh structure inside a mold to form a mesh footprint on the outside surface of the balloon;
removing the imprinted balloon from said mold;
attaching mesh to the surface of said imprinted balloon;
adding an overcoat to said mesh to affix said mesh to said surface of said imprinted balloon; thereby
providing a catheter balloon comprising a mesh-reinforced low compliant balloon material wherein said mesh is a highly oriented version of said balloon material.

17. A method according to claim 11 wherein said method comprises:
placing an ultra-high molecular weight fibrous mesh pre-form over balloon tubing in a mold;
blowing the inside of said balloon tubing to form a balloon shape and to fuse said mesh pre-form into the balloon tubing material; and
removing said balloon tubing with said fused mesh pre-form from said mold thereby
providing a catheter balloon comprising a mesh-reinforced low compliant balloon material wherein said mesh is a highly oriented version of said balloon material.

18. A method according to claim 17 wherein said mesh pre-form and said balloon tubing comprise a material selected from the group consisting of polyamide and polyethylene.

19. A method according to claim 11 wherein said method comprises:
injection molding a balloon pre-form with a mesh insert; and
blowing said balloon pre-form using a stretch blow molding process thereby
providing a catheter balloon comprising a mesh-reinforced low compliant balloon material wherein said mesh is a highly oriented version of said balloon material.

20. A method according to claim 19 wherein said balloon material is selected from the group consisting of polyamide, co-polyamide, aromatic polyamide, polyester, polyethylene and ultra-high molecular weight polyethylene (UHMWPE).
